(19) United States
(12) Patent Application Publication
(10) Pub. No.: US 2005/0036945 A1
(43) Pub. Date: Feb. 17, 2005

(54) Resorbable radiopaque markers and related medical implants

(76) Inventors: Kevin A. Thomas, San Diego, CA (US); L. Brian Starling, Golden, CO (US); James E. Stephan, Golden, CO (US); Kenneth K. Kleinhenz, Golden, CO (US)

Correspondence Address: Kenton R. Mullins;Stout, Uxa, Buyan & Mullins, LLP, 4 Venture, Suite 300, Irvine, CA, 92618, US

(21) Appl. No.: 10/762,204

(22) Filed: Jan. 20, 2004

Related U.S. Application Data
(60) Provisional application No. 60/441,040, filed on Jan. 17, 2003.

(51) Int. Cl. A61K 49/04

(52) U.S. Cl. 424/9.4; 424/426

(57) ABSTRACT

A resorbable medical implant is disclosed that includes one or more resorbable radiopaque markers. The resorbable radiopaque markers permit visualization of the location of the implant in a patient while reducing obstruction of tissue changes occurring in proximity to the implant. The resorbable radiopaque markers may include non-metallic and/or non-bone derived materials.

### CROSS-REFERENCE TO RELATED APPLICATIONS

This application claims the benefit of U.S. Provisional Application Ser. No. 60/441,040, filed Jan. 17, 2003, the entire contents of both of which are incorporated herein by reference.

### BACKGROUND OF THE INVENTION

1. Field of the Invention

The present invention relates generally to medical devices and, more particularly, to resorbable radiopaque markers used in resorbable medical implants.

2. Description of Related Art

Polymeric implant materials are generally not visible on radiographic images. In addition, polymeric materials typically do not obscure or interfere with other imaging techniques such as CT or MRI scans. The use of polymeric materials in medical implants is an advantage in certain applications, such as, for example, in fracture fixation or in cranial flap fixation in the craniomaxillofacial region where it may be desirable to image the underlying structures without artifacts from metallic implant devices.

Metallic radiopaque markers are used for various radiographic imaging studies, such as, for example, tantalum spherical beads. These metallic markers, however, may interfere with or prevent certain imaging techniques such as CT or MRI scans. Furthermore, metallic markers are not resorbable.

In a number of other applications, such as cement restrictors, graft containment mesh, spinal interbody devices, and spinal plate devices, it is often desirable to confirm placement of the device by radiography. It may be desirable to visualize the maintenance of the device position during the course of clinical healing, particularly in the case of spinal implants where loss of stability (as suggested by a change in the implant position) may require additional surgical intervention.

Thus, there remains a need for improved medical implant devices.

### SUMMARY OF THE INVENTION

The present invention provides an implant device that includes one or more resorbable radiopaque markers located in the implant which facilitates visualization of the implant and the markers when the implant is placed in a patient while reducing visual obstruction of tissues surrounding the implanted device.

In one embodiment, a resorbable implant comprises an implant body formed from a resorbable polymeric material; and a resorbable radiopaque marker formed from a non-metallic, non-bone derived material.

In another embodiment, a resorbable implant comprises an implant body that comprises a polylactide material; and a resorbable radiopaque marker formed from a non-metallic material located in the implant body to facilitate radiographic visualization of the implant when the implant is placed in a patient.

Any feature or combination of features described herein are included within the scope of the present invention provided that the features included in any such combination are not mutually inconsistent as will be apparent from the context, this specification, and the knowledge of one of ordinary skill in the art. For purposes of summarizing the present invention, certain aspects, advantages and novel features of the present invention have been described herein. Of course, it is to be understood that not necessarily all such aspects, advantages or features will be embodied in any particular embodiment of the present invention. Additional advantages and aspects of the present invention are apparent in the following detailed description and claims.

### BRIEF DESCRIPTION OF THE DRAWING

FIG. 1 is a perspective view of a resorbable implant with resorbable radiopaque markers located in the implant.

### DETAILED DESCRIPTION OF THE INVENTION

Reference will now be made in detail to certain embodiments of the invention, examples of which are illustrated in the accompanying drawings. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims.

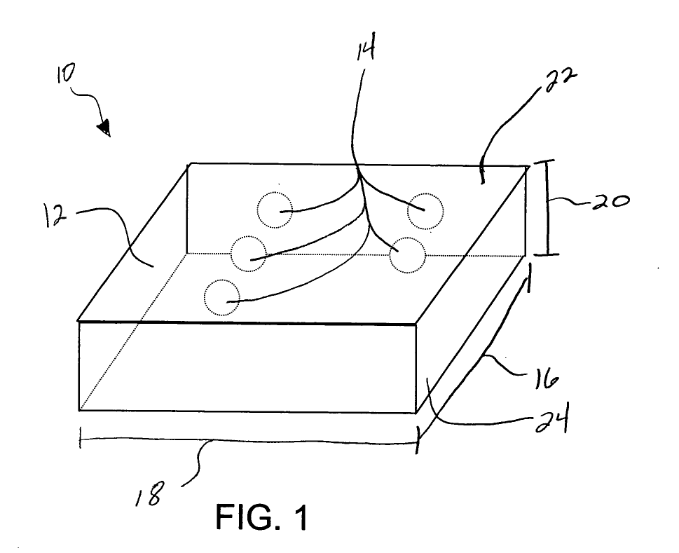

A resorbable implant 10 is illustrated in FIG. 1. The resorbable implant 10 comprises an implant body 12 and one or more resorbable radiopaque markers 14. The implant 10 is illustrated as having a length 16, a width 18, and a thickness 20. The implant 10 also includes a first surface 22, such as a top surface as illustrated, and a second surface 24, such as a bottom surface as illustrated.

The resorbable implant is configured to be implanted into a human or animal patient. Thus the implant is manufactured from a biocompatible material that does not cause a significant adverse reaction in the patient. In other words, the implant is manufactured from a biologically acceptable material. In certain embodiments, the implant body 12 is manufactured or formed from a resorbable polymeric material. The implant body 12 is radiolucent or is otherwise invisible when viewed radiographically when it is placed in a patient. The polymeric material used to form the implant body 12 may include, without limitation, polylactide, polyglycolide, derivatives and polymers/copolymers thereof, and mixtures thereof. In addition, the polymeric material may include other resorbable copolymers, or nonresorbable polymers. In additional embodiments, the implant device may be formed of a nonresorbable polymer or copolymer, or other nonmetallic material, such as allograft bone. The resorbable materials used in the manufacture of the implant may be obtained from manufacturers of such materials, or may be made using conventional procedures known to persons of skilled in the art.

The implant 10 may have a variety of configurations. As illustrated, the implant 10 may be in the form of a sheet or a plate. However, the implant 10 may also have a non-planar configuration, such as a curled configuration and the like. The implant 10 typically has a thickness 20 greater than the size of the radiopaque markers 14; however, in certain embodiments, the radiopaque markers 14 may protrude or be present at a surface of the implant. In certain embodiments, the implant 10 may have a thickness greater than about 1 mm, for example, 3 mm or greater. The implants also typically have a thickness which is suitable for the desired application to the patient, such as for repair of a defect or condition of a bone. The implant 10 may have any complex three-dimensional shape with various dimensions, such as a screw or other complex shape, that it is possible to manufacture through known polymeric thermoforming operations.

The implant 10 includes one or more resorbable radiopaque markers 14. In certain embodiments, such as the illustrated embodiment, the implant 10 includes a plurality of resorbable radiopaque markers 14. The marker or markers 14 may be distributed in the implant body 12 in a configuration that reduces visual obstruction of tissues surrounding the implant in the patient. The marker or markers 14 may be provided in a portion of the implant body, or in the entire implant body. In addition, markers 14 may be randomly or nonrandomly distributed in the implant body. In certain configurations in which the markers are nonrandomly distributed, the positional relationship of the implant to the target tissue may be more easily ascertained.

In addition, the markers 14 may be sized to reduce or prevent visual obstruction of the surrounding tissues. For example, the makers 14 may have a size less than about 2.0 mm. In certain embodiments, the markers 14 have sizes ranging from about 0.25 mm to about 3.0 mm, for example about 1.0 mm. The size of the marker may be one or more of the following: width, length, diameter, thickness, area, and volume. In at least one embodiment, the markers 14 are particles having substantially spherical shapes. As shown in the illustrated embodiment, the markers 14 may be entirely spherical. However, in additional embodiments, the markers may be particles have substantially non-spherical or aspherical configurations. In addition, the implant 10 may include two or more populations of radiopaque markers, each population having a different configuration. For example, the implant 10 may include a first population of spherical markers, and a second population of aspherical markers.

The markers 14 are radiopaque and resorbable in accordance with the illustrated embodiment disclosed herein. The marker or markers 14 may be made of a resorbable material that is resorbed, for example, after an expected healing period for the implantation or medical procedure. For example, the resorbable markers 14 may include a material that is resorbed by the patient after approximately 9 months from the implantation. In certain embodiments, the resorbable markers 14 may include a material that is resorbed by the patient approximately 12 months after implantation. In additional embodiments, the resorption of the markers may occur between about 16 and about 18 months after implantation. The resorption time or rate associated with the radiopaque markers may be altered by varying the composition of the marker by including or excluding in the composition materials known to have relatively faster or slower resorption rates. Resorption rates of various markers may be determined using conventional methods known to persons skilled in the art.

As disclosed herein, the resorbable radiopaque markers 14 can be used with various devices, such as implants, of sufficient size and geometry such that the device is larger than the radiopaque markers and therefore can contain the marker or markers. For example, as described above, the resorbable radiopaque markers may be spherical or nearly spherical with a diameter in the range of about 0.25 mm to about 3.0 mm. The resorbable radiopaque markers 14 can be used in any biocompatible device that has a geometric region (or thickness) sufficient for containing the marker or markers.

Examples of materials which may be useful in manufacturing the resorbable radiopaque markers disclosed herein can include one or more of barium sulfate, calcium phosphates, such as hydroxylapatite and tricalcium phosphate materials, calcium sulfates, such as “plaster of Paris,” barium apatites, other apatites known to occur in nature (for example by substitution of various materials for calcium in the hydroxylapatite material), calcium carbonates, calcium oxides, and any various combinations of the above materials. In one embodiment, the markers may comprise a combination of barium sulfate and tricalcium phosphate. In addition, the markers 14 can be fabricated in various compositions, for example 100% barium sulfate, 100% tricalcium phosphate, or in various combinations.

As discussed herein, appropriately sized markers (for example beads having a diameter of about 0.25 mm to about 3.0 mm) can be placed within various implant components fabricated from various materials as described.

In accordance with one aspect of the invention, materials for manufacturing the resorbable radiopaque markers disclosed herein can include, in addition or as an alternative to any of the above, (i) barium sulfate or substantial equivalent, and (ii) polymer additive components (PAC) otherwise known as polymer binder components or a polymeric binder composition, such as a combination including butyl stearate or substantial equivalent, canola oil or substantial equivalent; stable flake-S solidified oil or substantial equivalent, NA 860-000 or substantial equivalent, medium weight (MW) polyethylene (PE) or substantial equivalent, and hi flow polystyrene or substantial equivalent, and any various combinations of the above materials.

In certain embodiments, the markers may be formed of a barium sulfate injection molding formulation, wherein the feedstock formulation prepared during a thermal mixing (compounding) operation comprises a combination of 40-60 volume percent barium sulfate and 60-40 volume percent PAC.

For example, the compounded feedstock can comprise 50 volume percent each of barium sulfate and PAC, wherein the barium sulfate may have a powder density of about 4.4 g/cc and the PAC may have a density of about 1.0 g/cc. Thus, the 50 volume percent PAC equals 81.5 weight percent (w/o) and the 50 volume percent BA equals 18.5 w/o which equals a total of 100.0 w/o. In this example, a typical feedstock formulation for compounding can include:

1,000 g barium sulfate powder (Supplier F, Cat. No. B75)

227 g of PAC total

TABLE 1

| | | Weight | | Range of |
|---|---|---|---|---|
| | | percent | Amount | acceptability |
| PAC Binder Material | Supplier | (w/o) | (g) | (w/o) |
| Butyl Stearate | A | 5 | 11.35 | 0-10 |
| Solo 1000 Canola Oil | B | 25 | 56.75 | 20-30 |
| Stable Flake-S Solidified Oil | B | 20 | 45.40 | 15-25 |
| NA 860-000 | C | 25 | 56.75 | 20-30 |
| Medium MW PE | D | 20 | 45.20 | 15-25 |
| Hi Flow Polystyrene | E | 5 | 11.35 | 0-10 |
| Totals | 100 | 226.8 | | |

Supplier A: Kemestar, Humko Division, Astro Chemicals, Memphis TN
Supplier B: CT Custom Shortenings & Oils, Richmond VA
Supplier C: Equistar Chemicals, Morris IL
Supplier D: Phillips Petroleum, Polymers Division, Bartlesville OK
Supplier E: Monsanto Chemical Co., St. Louis MO or BASF Corporation, Mount Olive NJ
Supplier F: Fisher Scientific, Hanover Park IL

The above formulation for preparing feedstock for injection molding is but one example that is applicable to the requirement for molding spheres of barium sulfate. Mutsuddy in Ceramic Injection Molding, Mutsuddy, Beebhas C. and Ford, Renee G., Ceramic Injection Molding, Chapman & Hall, London, 1995. (ISBN 0 412 53810 5, describes a host of polymeric binder compositions many of which are appropriate for preparing barium sulfate feedstock. Mutsuddy also describes the importance of a proper rheology at molding temperatures for molding complex ceramic articles. These same principles may apply to the molding of barium sulfate spheres using state-of-the-art injection molding equipment and processing specifically geared to ceramic materials. Alternatively radiopaque beads can be made by other known material processing methods, such as extrusion, pressing and the like, which can further limit the amount of binder additives necessary to form beads to 10% by volume, or less.

In accordance with an aspect of the present invention, a process for fabricating barium sulfate spheres is reflected in the following steps:
1. Submicron barium sulfate powder (characterize and specify chemical analysis and monitor particle size and surface area);
2. Powder binding mix compounding (feedstock preparation);
3. Injection molding spheres;
4. Thermal debinding molded spheres and sintering barium sulfate spheres; and
5. Inspection.
   For the fabrication of sintered barium sulfate spheres, a thermal debinding operation can be used for removing the PAC in the molded spheres. The thermal debinding cycle can be integrated into the sintering cycle for the barium sulfate spheres. A typical thermal processing cycle includes both the thermal debinding of the PAC and sintering of the barium sulfate spheres.
   The following is an injection molding process overview:

As a preliminary step, tooling provides a mold cavity for a component and can include slides for three-dimensional features and multiple cavities for higher production quantities. Tool making can focus on making high quality molds for precision component, utilizing equipment such as EDM's (electrode discharge machining), milling machines, surface and centerless grinders, lathes and precision measuring instruments. Tooling for ceramic injection molding is comparable to plastic injection molding, with some cost-varying factors including grade of tool steel, number of cavities, use of slides, temperature control system, dimensional tolerances and surface finish required.

Mixing comprises making feedstock for the injection molding process. The mixing process starts with the selection of ceramic or metal powders which have the chemical composition desired to produce the final product. These powders are combined under heat, with a thermoplastic binder system. The mixture is then cooled to room temperature and granulated into pellets. These pellets, called “feedstock,” are injected into the final mold.

A molding machine of conventional commercial design made for example from the above tooling steps can accept feedstock. The feedstock is fed into the barrel of the machine where it is heated until it can flow freely. The material is then injected into the mold cavity through the sprues, runners and gates built into each tool. When the material cools sufficiently to hold its shape, the mold opens along the parting line and ejector pins push the component out. The machine then closes and the molding step is repeated. Runners, gates, and sprues are separated from the components.

De-binding can be used for larger parts to remove a portion of the thermoplastic binder from the component to facilitate sintering. For smaller parts, de-binding can be combined with sintering. De-binding can be accomplished by controlled heat application and/or controlled solvent system extraction. The de-binding step opens up microscopic passages within the component to assist the high temperature sintering process. After de-binding, an optimum amount of binder preferably remains to allow the component to maintain its shape during sintering.

A sintering step removes the remaining binder while facilitating movement of the powder particles. This allows the component to hold its original molded shape. Sintering can be continued until the part is a desired density and size as determined by the chosen powders and binders. Parts are cooled to room temperature before they are removed from the furnace. The fully sintered part retains its complex shape, through this highly controlled process. Close dimensional tolerances are preferably achieved.

As will be recognized by one skilled in the art, the debinding step and the sintering step are performed to remove all or substantially all of the binder material. The sintering step is generally performed at an elevated temperature to insure sintering as well as removal of any binder material not removed completely by the debinding step. Alternatively, these steps (debinding and sintering) may be performed such that some portion of the binder remains in the final product. Likewise, the debinding and sintering steps may also be adjusted to provide a wide range of density in the resulting radiopaque marker.

As one specific example, a spherical marker approximately 0.5 mm in diameter may be incorporated into a resorbable polylactide sheet, a resorbable polylactide plate, or other polylactide device that has a region having a thickness greater than 1 mm.

The resorbable implants disclosed herein may be configured to contain bone graft materials or designed to provide weak bony tissue support, among other things.

The incorporation of the resorbable radiopaque markers 14 into any of the implants 10, regardless or material, is possible for example by machining one or more appropriate sized holes, for example, a hole dimensioned to accommodate one or more markers 14, and inserting or press-fitting the marker or markers 14 into the-hole. Alternatively, the resorbable radiopaque markers 14 could be incorporated into implant devices during the manufacture of such devices (for example in the thermal forming manufacture or polymeric devices) or by other methods known by persons of skilled in the art.

The use of a radiographic marker, such as a bead, as disclosed herein, allows radiographic assessment of the implant's position without obscuring visualization of any other changes, such as tissue changes, surrounding the implant. In comparison, resorbable devices that incorporate a radiopaque material through the device to not permit such visualization. Metallic markers allow radiographic assessment of an implant's position but may interfere or prevent other imaging modalities such as CT or MRI scans.

Implant devices containing the resorbable radiopaque markers disclosed herein may be used to treat a variety of conditions. For example, the radiopaque markers may be provided in a cervical graft containment mesh device, such as resorbable mesh and screws; may be provided in an anterior cervical plate system, such as resorbable plates and screws, and other orthopedic applications. The implant devices disclosed herein may also be used to assess the stability of hip and knee arthroplasty implants, for example by incorporating the resorbable radiopaque markers into the articulating surfaces of these implants (made from nonresorbable polymeric materials). In spinal implant applications it may be desirable to have a marker that is both radiopaque and resorbable thereby reducing and avoiding problems associated with tissue surrounding metallic markers, as is conventionally practiced.

Thus, in accordance with the disclosure herein, an implant device includes a resorbable radiographic marker that allows assessment of the position of the device in which it is incorporated. The marker is of sufficient size to be visible on plain or conventional radiographs, yet small enough to be incorporated into various implant devices made from resorbable polymers or copolymers, nonresorbable polymers or copolymers, or other nonmetallic devices. The size of the marker or markers is also small enough that changes in the tissues surrounding the entire device are not substantially obscured. Since the marker is resorbable, the entire implant device, including the marker, is also resorbable. In the event that part or the entire device loosens or migrates from its initial implanted position, the fact that the marker is resorbable may obviate the need for a surgical intervention to remove the marker device or part of the implant device.

In one specific embodiment, a resorbable implant that is to be implanted in a patient comprises an implant body that includes a polylactide material, and one or more resorbable radiopaque markers formed from a non-metallic material. The non-metallic radiopaque marker is located in the implant body in a manner effective to facilitate radiographic visualization of the implant when the implant is placed in a patient.

The above-described embodiments have been provided by way of example, and the present invention is not limited to these examples. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the present invention is not intended to be limited by the disclosed embodiments, but is to be defined by reference to the appended claims.

1. An implant for implantation in a patient, comprising:
an implant body formed from a polymeric material; and
a resorbable radiopaque marker formed from a non-metallic, non-bone derived material and provided in the implant body to permit visualization of a location of the implant without obscuring visualization of changes surrounding the implant when the implant is placed in a patient, and which does not interfere with other imaging modalities such as CT or MRI scans.

2. The implant as set forth in claim 1, wherein the implant is resorbable and the implant body is formed from a resorbable polymeric material.

3. The implant as set forth in claim 2, wherein the resorbable radiopaque marker comprises at least one of calcium phosphate, hydroxylapatite, tricalcium phosphate, calcium sulfates, “plaster of Paris,” barium apatites, other apatites known to occur in nature, calcium carbonates, calcium oxides, and combinations of the above materials, with a suitable amount of polymer additive component to allow for formation of the marker.

4. The implant as set forth in claim 3, wherein the radiopaque marker comprises barium sulfate and polymer additive components.

5. The implant as set forth in claim 2, wherein the resorbable polymeric material comprises polylactide.

6. The implant as set forth in claim 2, wherein the resorbable polymeric material comprises at least one of biocompatible resorbable polymers, derivatives thereof, mixtures thereof, and copolymers thereof.

7. The implant as set forth in claim 6, wherein the resorbable polymeric material comprise at least one of polylactide, polyglycolide, derivatives thereof, mixtures thereof, and copolymers thereof.

8. The implant as set forth in claim 1, wherein the implant body comprises a nonresorbable polymer material.

9. The implant as set forth in claim 2, wherein the resorbable radiopaque marker comprises beads.

10. The implant as set forth in claim 2, wherein the implant body is formed as a sheet having a length, a width and a thickness.

11. The implant as set forth in claim 2, wherein the implant body has a complex three-dimensional shape.

12. The implant as set forth in claim 11, wherein the implant body is formed in the shape of a screw.

13. The implant as set forth in claim 2, comprising a plurality of resorbable radiopaque markers.

14. The implant as set forth in claim 2, wherein the resorbable radiopaque markers are sized to reduce visual obstruction of tissues surrounding the implant when the implant is placed in a patient.

15. The implant as set forth in claim 2, wherein the resorbable radiopaque markers are substantially spherical.

16. The implant as set forth in claim 15, wherein the resorbable radiopaque markers have a diameter of about 0.25 mm to about 3.0 mm.

17. The implant as set forth in claim 2, wherein the resorbable radiopaque marker is distributed in the implant body in a configuration effective to reduce visual obstruction of changes in tissues surrounding the implant when the implant is placed in a patient.

18. The implant as set forth in claim 2, wherein the resorbable radiopaque marker comprises barium sulfate and polymer additive components.

19. The implant as set forth in claim 18, wherein the resorbable radiopaque marker is manufactured using a sufficient amount of polymer additive components to allow processing into a bead form.

20. The implant as set forth in claim 18, wherein the resorbable radiopaque marker comprises 30-70 volume percent barium sulfate and 70-30 volume percent polymer additive components.

21. The implant as set forth in claim 20, wherein the resorbable radiopaque marker comprises particles formed using an injection molding process.

22. The implant as set forth in claim 2, wherein the resorbable radiopaque marker comprises particles formed using an injection molding process.

23. The implant as set forth in claim 18, wherein the resorbable radiopaque marker comprises about 10 volume percent or less polymer additive components.

24. The implant as set forth in claim 23, wherein the resorbable radiopaque marker is manufactured using a sufficient amount of polymer additive components to allow processing into a bead form.

25. The implant as set forth in claim 23, wherein the resorbable radiopaque marker comprises particles formed using an extrusion process.

26. The implant as set forth in claim 2, wherein the resorbable radiopaque marker is formed using a pressing process.

27. The implant as set forth in claim 23, wherein the resorbable radiopaque marker is formed using a pressing process.

28. The implant as set forth in claim 2, wherein the resorbable radiopaque marker comprises particles formed using a pressing process.

29. The implant as set forth in claim 18, wherein the polymer additive components comprise butyl stearate, canola oil; stable flake-S solidified oil, NA 860-000, medium weight polyethylene, and hi flow polystyrene.

30. A resorbable implant for implantation in a patient, comprising:
an implant body comprising a resorbable polymeric material having any complex three-dimensional shape; and
a plurality of radiopaque markers formed from a non-metallic material dispersed throughout at least a portion of the implant body to facilitate radiographic visualization of the implant when the implant is placed in a patient.

31. The implant as set forth in claim 30, wherein the resorbable polymeric material comprises at least one of biocompatible resorbable polymers, derivatives thereof, mixtures thereof, and copolymers thereof.

32. The implant as set forth in claim 31, wherein the resorbable polymeric material comprises at least one of polylactide, polyglycolide, derivatives thereof, mixtures thereof, and copolymers thereof.

33. The implant as set forth in claim 30, wherein the implant body is formed as a sheet having a length and a width.

34. The implant as set forth in claim 30, wherein the implant body is formed having a complex three-dimensional shape.

35. The implant as set forth in claim 30, wherein the resorbable radiopaque markers are beads.

36. The implant as set forth in claim 30, wherein the radiopaque markers are sized to reduce visual obstruction of tissues surrounding the implant when the implant is placed in a patient.

37. The implant as set forth in claim 30, wherein the radiopaque markers are substantially spherical.

38. The implant as set forth in claim 37, wherein the radiopaque markers have diameters of about 0.25 mm to about 3.0 mm.

39. The implant as set forth in claim 30, wherein the radiopaque markers are distributed in the implant body in a configuration effective to reduce visual obstruction of changes in tissues surrounding the implant when the implant is placed in a patient.

40. The implant as set forth in claim 30, wherein the radiopaque markers comprise barium sulfate and polymer additive components.

41. The implant as set forth in claim 40, wherein the radiopaque markers comprise 30-70 volume percent barium sulfate and 70-30 volume percent polymer additive components.

42. The implant as set forth in claim 40, wherein the polymer additive components comprise butyl stearate, canola oil; stable flake-S solidified oil, NA 860-000, medium weight polyethylene, and hi flow polystyrene.

43. The implant as set forth in claim 40, wherein the resorbable radiopaque marker has been manufactured using a sufficient amount of polymer additive components to allow processing into a bead form.

44. The implant as set forth in claim 43, wherein the resorbable radiopaque marker comprises about 10 volume percent or less polymer additive components.

45. The implant as set forth in 30, wherein the resorbable radiopaque marker comprises particles.

46. The implant as set forth in claim 45, wherein the resorbable radiopaque marker comprises at least one of calcium phosphate, hydroxylapatite, tricalcium phosphate, calcium sulfates, “plaster of Paris,” barium apatites, other apatites known to occur in nature, calcium carbonates, calcium oxides, and combinations of the above materials, with a suitable amount of polymer additive component to allow for formation of the particles.

47. The implant as set forth in claim 46, wherein the particles are beads.

48. The implant as set forth in claim 46, wherein the radiopaque marker comprises barium sulfate and polymer additive components.
